(19) United States
(12) Patent Application Publication
(10) Pub. No.: US 2026/0076880 A1
(43) Pub. Date: Mar. 19, 2026

(54) Hair Treatment Formulations, Dispensing Devices, And Methods

(71) Applicant: True Colors Chemistry, Inc., Solana Beach, CA (US)

(72) Inventors: Christopher Birchby, Solana Beach, CA (US); Sonia E. Chavez, Arlington, MA (US); Henry Harper, New Canaan, CT (US); Laura Muollo, Dracut, MA (US); Ju Rhyu, Beverly Hills, CA (US); John C. Warner, Wilmington, MA (US); Justin Whitfield, Billerica, MA (US)

(21) Appl. No.: 19/333,176

(22) Filed: Sep. 18, 2025

Related U.S. Application Data
(60) Provisional application No. 63/696,745, filed on Sep. 19, 2024.

(51) Int. Cl. A61K 8/06; A45D 19/02; A45D 34/04; A61K 8/19; A61K 8/22; A61K 8/92; A61K 8/9789

(52) U.S. Cl. CPC A61K 8/062; A45D 19/022; A45D 34/04; A61K 8/19; A61K 8/22; A61K 8/922; A61K 8/9789; A45D 2200/055; A45D 2200/058; A45D 2200/25; A61K 2800/52; A61K 2800/88

(57) ABSTRACT

Hair treatment formulations, dispensing devices, and methods provide safer, cleaner, less complicated alternatives to existing permanent hair dye kits for home or salon use. In some embodiments, a hair treatment formulation includes a dispersion and an aqueous solution. The dispersion includes a continuous phase including an oil as well as a dispersed phase including a pigment precursor and a catalyst for increasing an oxidation rate of the pigment precursor. The aqueous solution includes an oxidant for oxidizing the pigment precursor to a pigment. The dispersion and the aqueous solution form an emulsion for hair treatment upon their combination, the emulsion including the pigment. In some embodiments, the dispersion and the aqueous solution are distributed between compartments of a dispensing device for simultaneously combining the dispersion and the aqueous solution in a mixing tip to form the emulsion including the pigment through a dispensing action of the dispensing device.

### PRIORITY

This application claims the benefit of priority to U.S. Provisional Patent Application No. 63/696,745 , filed Sep. 19, 2024, which is incorporated by reference in its entirety into this application.

### BACKGROUND

A permanent hair dye kit for home use typically requires preparation that includes mixing a color cream and an activator to produce the hair dye of the kit. The color cream includes one or more dye precursors such as 1,4-diaminobenzene or 2,5-diaminotoluene and, optionally, one or more related couplers such as 1,3-diaminobenzene, 3-aminophenol, resorcinol, or the like. The activator includes an oxidant such as hydrogen peroxide in a greater concentration than that in household hydrogen peroxide. In addition, either the color cream or the activator can include ammonia, which provides, inter alia, basic reaction conditions during preparation. When the color cream is mixed with the activator, a dye precursor of the color cream is oxidized by the oxidant of the activator to produce a quinone diimine, which then reacts with another dye precursor or a coupler to produce a leuco-dye adduct that ultimately yields the hair dye upon oxidation of the leuco-dye adduct.

While less harmful formulations in permanent hair dye kits such as the foregoing color cream and activator have been in use since the 1970s, the dye precursors, couplers, and resulting hair dyes still pose health risks from preparation through application. Preparation of such hair dyes can be messy and time consuming, thereby adding contact opportunities with the dye precursors and couplers as well as overall exposure time to that already necessitated by the application of the hair dyes. In addition, preparation of such hair dyes is prone to error. What is needed is a safer, cleaner, less complicated alternative to existing permanent hair dye kits for at home or salon use.

Disclosed herein are hair treatment formulations, dispensing devices, and methods that address the foregoing.

### SUMMARY

Disclosed herein is a hair treatment formulation including, in some embodiments, a dispersion and an aqueous solution. The dispersion includes a continuous phase including one or more oils and a dispersed phase including a pigment precursor and one or more transition metal catalysts for increasing an oxidation rate of the pigment precursor. The aqueous solution includes an oxidant for oxidizing the pigment precursor to a pigment. The dispersion and the aqueous solution form an oil-in-water emulsion for hair treatment upon combination with each other, the emulsion including the pigment.

In some embodiments, the one or more oils are selected from coconut oil, fractionated coconut oil, olive oil, argan oil, avocado oil, jojoba oil, sweet almond oil, grapeseed oil, baobab oil, castor oil, macadamia nut oil, marula oil, and sea buckthorn oil.

In some embodiments, the pigment precursor is present in the dispersed phase as a constituent of Mucuna pruriens seed powder or an extract thereof.

In some embodiments, the one or more transition metals are selected from manganese and iron.

In some embodiments, the dispersed phase of the dispersion further includes one or more stabilizers for keeping the dispersed phase dispersed in the continuous phase. The one or more stabilizers are selected from dextrin palmitate, sucrose laurate, and dibutyl lauroyl glutamide.

In some embodiments, the dispersed phase of the dispersion further includes one or more pH-modifying agents in a concentration suitable for modifying a pH of an aqueous phase of the emulsion to be greater than pH 7 upon combination of the dispersion and the aqueous solution.

In some embodiments, the oxidant is hydrogen peroxide.

In some embodiments, the dispersion, the aqueous solution, or both the dispersion and the aqueous solution independently include one or more preservatives selected from phenethyl alcohol, sodium benzoate, gluconolactone, potassium sorbate, sodium anisate, sodium phytate, aspen bark extract, willow bark extract, leucidal liquid, propanediol, pentylene glycol, glycerin, ethylhexyl glycerin, caprylyl glycol, and glyceryl caprylate-caprate.

Also disclosed herein is a hair treatment kit including a dispensing device and a hair treatment formulation disposed in the dispensing device. The dispensing device includes a dispersion of the hair treatment formulation in a first compartment of the dispensing device and an aqueous solution of the hair treatment formulation in a second compartment of the dispensing device. The dispersion includes a continuous phase including one or more oils and a dispersed phase including a pigment precursor and one or more transition metal catalysts for increasing an oxidation rate of the pigment precursor. The aqueous solution includes an oxidant for oxidizing the pigment precursor to a pigment. The dispersion and the aqueous solution form an oil-in-water emulsion for hair treatment upon dispensation from the dispensing device and combination with each other, the emulsion including the pigment.

In some embodiments, the one or more oils are selected from coconut oil, fractionated coconut oil, olive oil, argan oil, avocado oil, jojoba oil, sweet almond oil, grapeseed oil, baobab oil, castor oil, macadamia nut oil, marula oil, and sea buckthorn oil.

In some embodiments, the pigment precursor is present in the dispersed phase as a constituent of Mucuna pruriens seed powder or an extract thereof.

In some embodiments, the one or more transition metal catalysts respectively include one or more transition metals selected from those in a range of the Periodic Table of the Elements defined by Groups 7-12 and Periods 4 and 5.

In some embodiments, the dispersed phase of the dispersion further includes one or more stabilizers for keeping the dispersed phase dispersed in the continuous phase. The one or more stabilizers are selected from dextrin palmitate, sucrose laurate, and dibutyl lauroyl glutamide.

In some embodiments, the dispersed phase of the dispersion further includes one or more pH-modifying agents in a concentration suitable for modifying a pH of an aqueous phase of the emulsion to be greater than pH 7 upon combination of the dispersion and the aqueous solution.

In some embodiments, the oxidant is hydrogen peroxide.

In some embodiments, the dispensing device further includes a mixing tip to which each compartment of the first and second compartments is fluidly connected in a ready-to-dispense state of the dispensing device. Dispensation from the dispensing device through the mixing tip combines the dispersion and the aqueous solution to form the emulsion.

In some embodiments, the dispensing device is a double-barreled syringe. The first compartment of the dispensing device corresponds to a first barrel chamber defined by a first barrel of the syringe. The second compartment of the dispensing device corresponds to a second barrel chamber defined by a second barrel of the syringe.

In some embodiments, the dispensing device is a double-compartmented tube or bag. Each compartment of the first and second compartments is defined by an external piece substantially independent of the other. Each compartment of the first and second compartments is also defined by an internal piece dividing the first and second compartments.

In some embodiments, the dispensing device is a double-compartmented tube or bag. One compartment of the first and second compartments is disposed in the other compartment of the first and second compartments.

In some embodiments, the hair treatment kit further includes one or more additional components selected from a package of chelating shampoo for removing at least transition metals from hair prior to applying the emulsion, a tint brush for applying the emulsion, and instructions for using the hair treatment kit.

These and other features of the concepts provided herein will become more apparent to those of skill in the art in view of the accompanying drawings and following description, which describe particular embodiments of such concepts in greater detail.

### DRAWINGS

FIG. 1 illustrates oxidation of a pigment precursor of the hair treatment formulation to a pigment in accordance with some embodiments.

FIG. 2 illustrates a double-barreled syringe with a mixing tip as a dispensing device in accordance with some embodiments, notably including the hair treatment formulation.

FIG. 3 illustrates a double-compartmented bag with a mixing tip as a dispensing device in accordance with some embodiments.

FIG. 4 illustrates a double-compartmented tube with a mixing tip as a dispensing device in accordance with some embodiments.

FIG. 5 illustrates the double-compartmented tube from a dispensing end including the mixing tip in accordance with some embodiments.

FIG. 6 illustrates the double-compartmented tube from a sealed end opposite the dispensing end of the double-compartmented tube in accordance with some embodiments, notably prior to sealing the sealed end of the double-compartmented tube.

FIG. 7 illustrates a double-compartmented pouch with a tear-off opening strip in accordance with some embodiments.

FIG. 8 illustrates the double-compartmented pouch with the tear-off opening strip in accordance with some other embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Any organic compound specified as its free acid, free base, or salt, as the case might be, is not limited to the specified free acid, free base, or salt. Indeed, any organic compound specified as its free acid can instead be a salt specified by its parent compound or a conjugate base thereof and a counterion of an alkali metal such as sodium or potassium; an alkaline earth metal such as calcium or magnesium; or the like. Any organic compound specified as its free base can instead be a salt specified by its parent compound or a conjugate acid thereof and a counterion of a halide such as chlorine, bromine, or iodine; an oxyanion such as carbonate, nitrate, sulfate, or phosphate; a carboxylate such as formate, acetate, or propionate; hydroxide; or the like. Any organic compound specified as a salt can instead be another salt in accordance with the foregoing; a free acid of the specified salt; or a free base of the specified salt.

Unless defined otherwise, all technical and scientific terms used herein have the same meaning as commonly understood by those of ordinary skill in the art.

As set forth above, a safer, cleaner, less complicated alternative to existing permanent hair dye kits for at home or salon use is needed. Disclosed herein are hair treatment formulations, dispensing devices, and methods that address the foregoing, as below.

### Hair Treatment Formulations

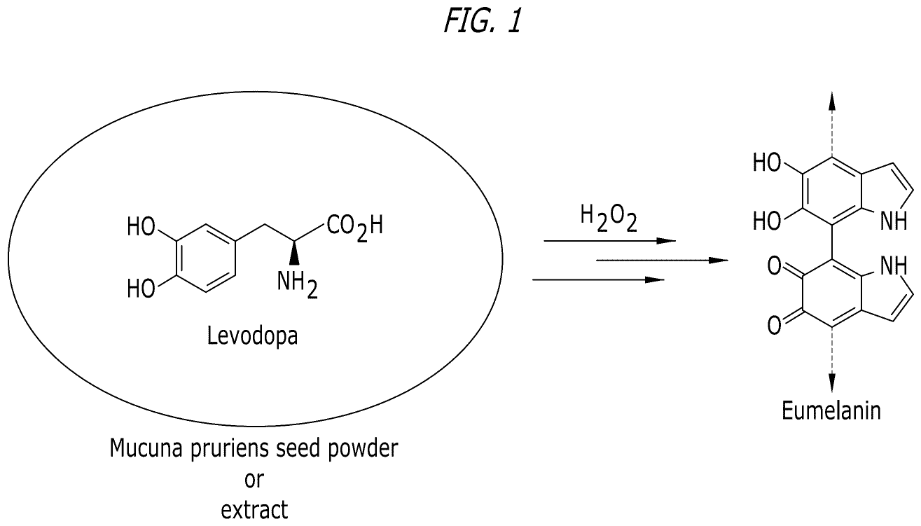

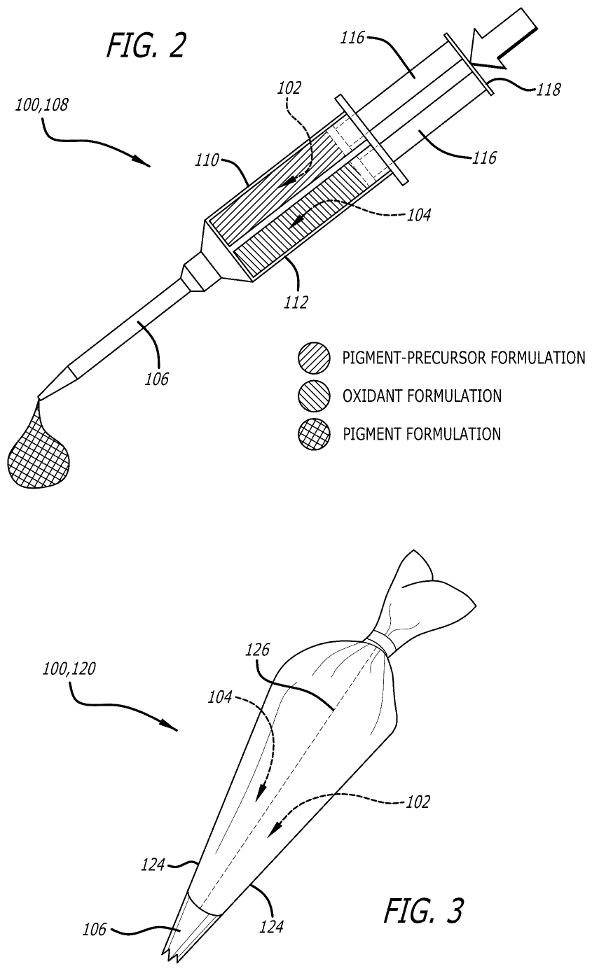

FIGS. 1 and 2 illustrate aspects of the hair treatment formulation in accordance with some embodiments.

The hair treatment formulation includes a pigment-precursor formulation and an oxidant formulation, which are configured to form a pigment formulation for hair treatment upon their combination, for example, as shown in FIG. 2. The pigment-precursor formulation includes a pigment precursor and the oxidant formulation includes an oxidant such that oxidation of the pigment precursor commences upon combination of the pigment-precursor formulation and the oxidant formulation, thereby forming a pigment in the pigment formulation for imparting or restoring color to hair in accordance with the hair treatment formulation.

The pigment-precursor formulation can be an oil-based dispersion and the oxidant formulation can be an aqueous solution as set forth in detail below. However, it should be understood that the inverse is also possible in which the pigment-precursor formulation is an aqueous solution and the oxidant formulation is an oil-based dispersion, albeit with some modifications to that set forth below. For example, when the pigment-precursor formulation is an aqueous solution, the pigment-precursor formulation can be acidic to enhance solubility of the pigment precursor (e.g., levodopa) in the aqueous solution. That, and when the oxidant formulation is an oil-based dispersion, the oxidant can be a solid such as urea-hydrogen peroxide (“UHP”) dispersed throughout the oxidant formulation. Additional base such as the sodium bicarbonate or sodium carbonate set forth below can be added as needed to create alkaline conditions in the pigment formulation upon combination of the pigment-precursor formulation and the oxidant formulation. Whether the pigment-precursor formulation is an oil-based dispersion and the oxidant formulation is an aqueous solution or vice versa, the pigment-precursor formulation and the oxidant formulation are configured to form an emulsion (e.g., an oil-in-water emulsion or a water-in-oil emulsion) as the pigment formulation for hair treatment upon their combination.

Continuing with the pigment-precursor formulation as the oil-based dispersion and the oxidant formulation as the aqueous solution, but with the understanding that the pigment-precursor formulation and the oxidant formulation are not limited to such mixtures, the pigment-precursor formulation includes the pigment precursor and the oxidant formulation includes the oxidant such that oxidation of the pigment precursor commences upon formation of the emulsion, thereby forming the pigment in the emulsion for imparting or restoring color to hair in accordance with the hair treatment formulation. Indeed, as shown in FIG. 1, the pigment precursor in the pigment-precursor formulation can include levodopa, the oxidant in the oxidant formulation can include hydrogen peroxide, and the pigment of the pigment formulation can include eumelanin for imparting or restoring color to hair. However, it should be understood that the pigment precursor and the oxidant can vary as set forth in detail below.

The oil-based dispersion includes a continuous phase and a dispersed phase dispersed in the continuous phase. The continuous phase of the oil-based dispersion includes one or more oils. The dispersed phase of the oil-based dispersion includes the pigment precursor and, optionally, one or more transition metal catalysts for increasing an oxidation rate of the pigment precursor. Being that an oxidant such as hydrogen peroxide is not optimally shelf stable above about pH 5, the dispersed phase of the oil-based dispersion preferably includes one or more pH-modifying agents instead of the aqueous solution for basifying the aqueous phase of the emulsion formed upon combination of the oil-based dispersion and the aqueous solution. That said, the aqueous solution can include its own pH-modifying agent(s) including, but not limited to, citric acid for acidifying the aqueous solution to about pH 5 or lower for shelf stability prior to the foregoing combination with the oil-based dispersion. Lastly, the dispersed phase of the oil-based dispersion can include one or more stabilizers for keeping the dispersed phase dispersed in the continuous phase.

The one or more oils are selected from at least coconut oil, fractionated coconut oil, which is also known as medium-chain triglyceride (“MCT”) oil, olive oil, argan oil, avocado oil, jojoba oil, sweet almond oil, grapeseed oil, baobab oil, castor oil, macadamia nut oil, marula oil, and sea buckthorn oil.

The pigment precursor can be any catecholamine expected to form one or more 5,6-dihydroxyindoles en route to a polymer as the pigment. As shown in FIG. 1, such a catecholamine includes, but is not limited to, levodopa, which forms 5,6-dihydroxyindole (“DHI”) and 5,6-dihydroxyindole 2-carboxylic acid (“DHICA”) en route to eumelanin as the pigment. Levodopa naturally occurs in Mucuna pruriens seeds up to at least about 6% by dry weight, so levodopa can be present in the dispersed phase of the oil-based dispersion as a constituent of M. pruriens seed powder, a constituent of an extract of M. pruriens seeds, or a substantially pure form of levodopa derived from M. pruriens seeds. However, it should be understood that levodopa can additionally or alternatively be sourced from any part of M. pruriens as well as any other natural (or genetically modified) plant having tyrosinase including, but not limited to, Stizolobium deeringianum or Vicia faba. Further, levodopa can even be sourced from a synthetic source. Additionally or alternatively, the pigment precursor can include any one or more oxidation intermediates of the catecholamine en route to the pigment such as any one or more stable intermediates of melanogenesis selected from at least leucodopachrome, dopachrome, DHI, DHICA, or the like. Notably, the pigment precursor can additionally or alternatively include any one or more stable intermediates en route to pheomelanin selected from at least 5-S-cysteinyldopa and 1,4-benzothiazinyl-alanine.

As set forth below, a combination or admixture of constituents including the pigment precursor, the one or more transition metal catalysts, and the one or more pH-modifying agents can be mixed into the one or more oils, optionally, already including the one or more stabilizers. With the understanding that M. pruriens seeds include up to at least about 6% levodopa by dry weight, such an admixture can include M. pruriens seed powder or an extract thereof in a concentration of at least about 20%, 30%, 40%, 50%, 60%, 70%, or 80% by weight inclusive of any intervening concentrations defined by the so-called ones place. Alternatively, the admixture can include M. pruriens seed powder or an extract thereof in a concentration of no more than about 80%, 70%, 60%, 50%, 40%, 30%, or 20% by weight inclusive of any intervening concentrations defined by the so-called ones place. As such, the admixture can include M. pruriens seed powder or an extract thereof in a concentration of at least about 20% and no more than about 80% by weight, including at least about 30% and no more than about 70% by weight, such as at least about 40% and no more than about 60% by weight, for example, at least about 45% and no more than about 55% by weight. Being that levodopa can be sourced from any natural (or genetically modified) plant having tyrosinase, however, the admixture can alternatively or additionally include a powder or extract of such a plant (or part thereof) in a concentration within one of the foregoing concentration ranges. It should be understood that the concentration of levodopa vehicle (e.g., the M. pruriens seed powder or extract thereof) in the admixture can vary in accordance with the concentration of levodopa in the levodopa vehicle. Indeed, lower concentrations of levodopa in the levodopa vehicle (e.g., <5% levodopa in the M. pruriens seed powder or extract thereof) lead to some of the higher concentrations given for the levodopa vehicle in the admixture, and higher concentrations of levodopa in the levodopa vehicle (e.g., >5% levodopa in the M. pruriens seed powder or extract thereof) lead to some of the lower concentrations given for the levodopa vehicle in the admixture. In step with the latter, substantially pure forms of levodopa lead to lower concentrations of levodopa in the admixture.

When present, the one or more transition metal catalysts respectively include one or more transition metals selected from those in a range of the Periodic Table of the Elements defined by the d-Block and, optionally, the f-Block, which transition metals are often considered inner transition metals. The one or more transition metals can be further selected from those in a range of the Periodic Table of the Elements defined by Groups 3-12 and Periods 4-6, including Groups 4-12 and Periods 4 and 5, such as Groups 6-12 and Periods 4 and 5, for example, Groups 7-12 and Period 4, or some combination of the foregoing Groups and Periods. For example, the one or more transition metals can be selected from titanium, chromium, molybdenum, manganese, iron, cobalt, nickel, copper, silver, and zinc. Notably, such one or more transition metals can assume any of a number of useful oxidation states for increasing the oxidation rate of the pigment precursor. Continuing with the manganese and iron of the foregoing example, the one or more transition metal catalysts can be selected from manganese gluconate and iron gluconate, the transition metal of each of which is in its +2-oxidation state.

As set forth below, a combination or admixture of constituents including the pigment precursor, the one or more transition metal catalysts, and the one or more pH-modifying agents can be mixed into the one or more oils, optionally, already including the one or more stabilizers. Such an admixture can include the one or more transition metal catalysts in a concentration of at least about 1%, 5%, 10%, 15%, 20%, or 25% by weight inclusive of any intervening concentrations defined by the so-called ones place. Further, the admixture can include the one or more transition metal catalysts in a concentration of at least about 0.1%, 0.2%, 0.3%, 0.4%, 0.5%, 0.6%, 0.7%, 0.8%, or 0.9% by weight inclusive of any intervening concentrations defined by the so-called hundredths place. Alternatively, the admixture can include the one or more transition metal catalysts in a concentration of no more than about 25%, 20%, 15%, 10%, 5%, or 1%, by weight inclusive of any intervening concentrations defined by the so-called ones place. Included in the foregoing, is a concentration of the one or more transition metal catalysts in the admixture of no more than about 0.9%, 0.8%, 0.7%, 0.6%, 0.5%, 0.4%, 0.3%, 0.2%, or 0.1% by weight inclusive of any intervening concentrations defined by the so-called hundredths place. As such, the admixture can include the one or more transition metal catalysts in a concentration of at least about 0.1% and no more than about 25% by weight, including at least about 0.5% and no more than about 20% by weight, such as at least about 1% and no more than about 15% by weight, for example, at least about 5% and no more than about 10% by weight.

The one or more pH-modifying agents are selected from organic bases and inorganic bases, any of which can be present as part of a buffering system with its conjugate acid(s). For example, an organic base can include a citrate such as sodium citrate or a lactate such as sodium lactate. For example, an inorganic base can include a hydroxide such as ammonium hydroxide, a carbonate such as sodium bicarbonate or sodium carbonate, or a phosphate such as sodium dihydrogen phosphate, sodium hydrogen phosphate, or trisodium phosphate. Continuing with the intervening example, the sodium bicarbonate and sodium carbonate can be part of a carbonate buffering system for modifying the pH of the aqueous phase of the emulsion upon combination of the oil-based dispersion and the aqueous solution as well as maintaining the pH of the aqueous phase of the emulsion so modified. Notably, the one or more pH-modifying agents are in a concentration in the oil-based dispersion suitable for modifying the pH of the aqueous phase of the emulsion upon combination of the oil-based dispersion and the aqueous solution. Notably, the pH of the aqueous phase of the emulsion can be greater than about pH 7 upon combination of the oil-based dispersion and the aqueous solution for effectuating oxidation of the pigment precursor.

As set forth below, a combination or admixture of constituents including the pigment precursor, the one or more transition metal catalysts, and the one or more pH-modifying agents can be mixed into the one or more oils, optionally, already including the one or more stabilizers. Such an admixture can include the one or more pH-modifying agents in a concentration of at least about 20%, 30%, 40%, 50%, 60%, 70%, or 80% by weight inclusive of any intervening concentrations defined by the so-called ones place. Alternatively, the admixture can include the one or more pH-modifying agents in a concentration of no more than about 80%, 70%, 60%, 50%, 40%, 30%, or 20% by weight inclusive of any intervening concentrations defined by the so-called ones place. As such, the admixture can include the one or more pH-modifying agents in a concentration of at least about 20% and no more than about 80% by weight, including at least about 30% and no more than about 70% by weight, such as at least about 40% and no more than about 60% by weight, for example, at least about 45% and no more than about 55% by weight.

When present, the one or more stabilizers are selected from at least dextrin palmitate; esterified sugars such as glucose laurate, sucrose laurate, and sucrose stearate; fatty-acid esters such as cetyl palmitate, cetyl ethylhexanoate, cetyl stearate, stearyl ethylhexanoate, stearyl stearate; and dibutyl lauroyl glutamide.

As set forth below, the one or more stabilizers can be mixed into the one or more oils, thereby forming the nascent oil-based dispersion of the one or more stabilizers in the one or more oils. The nascent oil-based dispersion can include the one or more stabilizers in a concentration of at least about 1%, 5%, 10%, 15%, 20%, or 25% by weight inclusive of any intervening concentrations defined by the so-called ones place. Alternatively, the nascent oil-based dispersion can include the one or more stabilizers in a concentration of no more than about 25%, 20%, 15%, 10%, 5%, or 1% by weight inclusive of any intervening concentrations defined by the so-called ones place. As such, the nascent oil-based dispersion can include the one or more stabilizers in a concentration of at least about 1% and no more than about 25% by weight, including at least about 1% and no more than about 20% by weight, such as at least about 1% and no more than about 15% by weight, for example, at least about 5% and no more than about 10% by weight.

As set forth below, a combination or admixture of constituents including the pigment precursor, the one or more transition metal catalysts, and the one or more pH-modifying agents can be mixed into the one or more oils including the one or more stabilizers, namely, the nascent oil-based dispersion, thereby forming the oil-based dispersion of the hair treatment formulation. The admixture can have a concentration of at least about 20%, 30%, 40%, 50%, 60%, 70%, or 80% by weight in the oil-based dispersion inclusive of any intervening concentrations defined by the so-called ones place. Alternatively, the admixture can have a concentration of no more than about 80%, 70%, 60%, 50%, 40%, 30%, or 20% by weight in the oil-based dispersion inclusive of any intervening concentrations defined by the so-called ones place. As such, the admixture can have a concentration in the oil-based dispersion of at least about 20% and no more than about 80% by weight, including at least about 30% and no more than about 70% by weight, such as at least about 40% and no more than about 60% by weight, for example, at least about 45% and no more than about 55% by weight.

The aqueous solution includes water and an oxidant dissolved therein for oxidizing the pigment precursor to the pigment. The oxidant can be hydrogen peroxide as shown in FIG. 1. Additionally or alternatively, the oxidant can include any one or more oxidants selected from organic oxidants and inorganic oxidants other than hydrogen peroxide. An organic oxidant can include, for example, UHP, tert-butyl hydrogen peroxide, or 4-hydroxy-2,2,6,6-tetramethylpiperidin-1-oxyl (“TEMPO”). An inorganic oxidant can include, for example, a percarbonate such as sodium percarbonate, an iodate such as sodium iodate or potassium iodate, a perborate such as sodium perborate, a peroxydisulfate such as ammonium peroxydisulfate, sodium peroxydisulfate, or potassium peroxydisulfate, a bromate such as sodium bromate or potassium bromate, a hypochlorite such as sodium hypochlorite, a periodate such as sodium periodate or potassium periodate.

As set forth below, the oxidant can be mixed into water before the one or more viscosity modifiers. When such an aqueous solution includes 3% aqueous hydrogen peroxide as the oxidant, the aqueous solution can include the 3% aqueous hydrogen peroxide in a concentration of at least about 20%, 30%, 40%, 50%, 60%, 70%, or 80% by weight inclusive of any intervening concentrations defined by the so-called ones place. Alternatively, the aqueous solution can include the 3% aqueous hydrogen peroxide in a concentration of no more than about 80%, 70%, 60%, 50%, 40%, 30%, or 20% by weight inclusive of any intervening concentrations defined by the so-called ones place. As such, the aqueous solution can include the 3% aqueous hydrogen peroxide in a concentration of at least about 20% and no more than about 80% by weight, including at least about 20% and no more than about 70% by weight, such as at least about 20% and no more than about 60% by weight, for example, at least about 30% and no more than about 50% by weight.

The aqueous solution can further include one or more viscosity modifiers in a concentration suitable for modifying a viscosity of the aqueous solution. Indeed, the viscosity of the aqueous solution can be coordinated with that of the oil-based dispersion to facilitate combination of the oil-based dispersion and the aqueous solution in the mixing tip 106 of the dispensing device 100 to form the emulsion. When present, the one or more viscosity modifiers are selected from at least one or more gums including konjac gum, sclerotium gum, xanthan gum, carrageenan gum, guar gum, locust bean gum, and chemically modified forms thereof such as hydroxypropyl guar. Additionally or alternatively, the one or more viscosity modifiers are selected from at least one or more cellulosics including hydroxypropyl methylcellulose or some other cellulose ether.

As set forth below, the one or more viscosity modifiers can be mixed into the aqueous solution including the oxidant, thereby forming the aqueous solution of the hair treatment formulation. The one or more viscosity modifiers can have a concentration of at least about 0.5%, 1.0%, 2.0%, 3.0%, 4.0%, or 5.0% by weight in the aqueous solution of the hair formulation inclusive of any intervening concentrations defined by the so-called tenths place. Alternatively, the one or more viscosity modifiers can have a concentration of no more than about 5.0%, 4.0%, 3.0%, 2.0%, 1.0 or 0.5% by weight in the aqueous solution of the hair formulation inclusive of any intervening concentrations defined by the so-called tenths place. As such, the one or more viscosity modifiers can have a concentration in the aqueous solution of the hair formulation of at least about 0.5% and no more than about 5% by weight, including at least about 0.5% and no more than about 3% by weight, such as at least about 0.5% and no more than about 2% by weight, for example, at least about 0.5% and no more than about 1% by weight. Notably, depending upon the species of the one or viscosity modifiers, concentrations of the one or more viscosity modifiers more than about 1% can form gels instead of solutions.

The oil-based dispersion, the aqueous solution, or both the oil-based dispersion and the aqueous solution can independently include one or more phase-transfer catalysts. Such one or more phase-transfer catalysts facilitate ion shuttling between the oil-based dispersion and the aqueous solution, mixing the oil-based dispersion and the aqueous solution, particularly, when a phase-transfer catalyst also functions as a surfactant, or both upon combination of the oil-based dispersion and the aqueous solution. When present, the one or more phase-transfer catalysts are selected from at least quaternary ammonium salts, which, in turn, include at least alkyltrimethylammonium salts such as cetyltrimethylammonium chloride and docosyltrimethylammonium chloride; dialkyldimethylammonium salts such as dicetyldimethylammonium chloride and distearyldimethylammonium chloride; and alkylbenzyldimethylammonium salts, which are also known as benzalkonium salts, such as dodecylbenzalkonium chloride, tetradecylbenzalkonium chloride, hexadecylbenzalkonium chloride, and stearalkonium chloride.

The oil-based dispersion, the aqueous solution, or both the oil-based dispersion and the aqueous solution can independently include one or more emulsifiers to reduce the surface tension between the oil-based dispersion and the aqueous solution allowing them to mix and form the emulsion of the pigment formulation.

The oil-based dispersion, the aqueous solution, or both the oil-based dispersion and the aqueous solution can independently include one or more preservatives. When present, the one or more preservatives are selected from at least phenethyl alcohol, sodium benzoate, gluconolactone, potassium sorbate, sodium anisate, sodium phytate, aspen bark extract, willow bark extract, leucidal liquid, propanediol, pentylene glycol, glycerin, ethylhexyl glycerin, caprylyl glycol, and glyceryl caprylate-caprate. Like that set forth above, any preservative of the foregoing preservatives specified as its free acid, free base, or salt, as the case might be, is not limited to the specified free acid, free base, or salt. Any preservative of the foregoing preservatives specified as its free acid can instead be a salt specified by its parent compound or a conjugate base thereof and a counterion of an alkali metal such as sodium or potassium; an alkaline earth metal such as calcium or magnesium; or the like. Any preservative of the foregoing preservatives specified as its free base can instead be a salt specified by its parent compound or a conjugate acid thereof and a counterion of a halide such as chlorine, bromine, or iodine; an oxyanion such as carbonate, nitrate, sulfate, or phosphate; a carboxylate such as formate, acetate, or propionate; hydroxide; or the like. Any preservative of the foregoing preservatives specified as a salt can instead be another salt in accordance with the foregoing; a free acid of the specified salt; or a free base of the specified salt. For example, the one or more preservatives can be a combination of phenethyl alcohol, glycerin, and benzoic acid.

The oil-based dispersion, the aqueous solution, or both the oil-based dispersion and the aqueous solution can independently include one or more haircare ingredients that impart haircare benefits. When present, the one or more haircare ingredients are selected from at least tea tree oil, rosemary oil, panthenol, various plant-derived acids including at least lactic acid and salicylic acid, retinol, various peptides, and various antioxidants.

The oil-based dispersion, the aqueous solution, or both the oil-based dispersion and the aqueous solution can independently include one or more hair-penetration ingredients that improve penetration of various ingredients into hair. When present, the one or more hair-penetration ingredients are selected from at least alcohols including diols such as glycols and triols such as glycerin, fatty acids, amino acids, peptides, hydrolyzed proteins, and various surfactants.

The oil-based dispersion, the aqueous solution, or both the oil-based dispersion and the aqueous solution can independently include one or more emollients that sooth and soften both scalp and hair. When present, such emollients are typically part of the oil-based dispersion. The one or more emollients are selected from at least fatty acid esters, which include isoamyl laurate, various triglycerides, coco-caprylate, various isostearates, or some combination thereof, as well as waxy esters, which include various behenates, various palmitates, various stearates, or some combination thereof. The foregoing fatty acid and waxy esters improve penetration of the ingredients into the hair, particularly those fatty acids and esters having lower molecular weights. The fatty acid and waxy esters also influence the texture or feel of the hair after treatment, particularly those fatty acids and esters having higher molecular weights. Notably, the stability and structure of the oil-based dispersion can also be enhanced by the fatty acid and waxy esters.

Notably, as with the pigment-precursor levodopa that naturally occurs in M. pruriens seeds, constituents of the hair treatment formulation are preferably sourced from safe, non-toxic natural sources or at least found in such natural sources, thereby ensuring the hair treatment formulation is a safer alternative to existing permanent hair dye kits for at home or salon use. Should one or more constituents of the hair treatment formulation not be found in such natural sources, the one or more constituents are preferably previously reviewed and found safe by the Cosmetic Ingredient Review of Washington D.C.

### Dispensing Devices

FIGS. 2, 3, 4-6, 7, and 8 illustrate various dispensing devices for dispensing the hair treatment formulation in accordance with some embodiments.

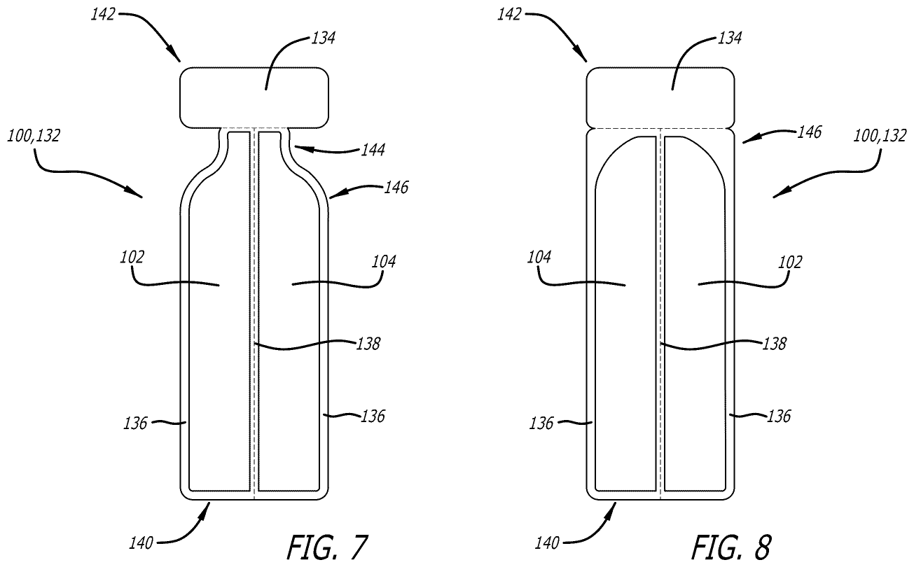

As shown, each dispensing device of the various dispensing devices is a dual- or double-compartmented dispensing device 100 including a first compartment 102 and a second compartment 104. With the exception of the dispensing device 132 of FIGS. 7 and 8, the foregoing dispensing devices also include a mixing tip 106 to which each compartment of the first and second compartments 102 and 104 is fluidly connected in at least a ready-to-dispense state of the dispensing device 100. Notably, the mixing tip 106 need not be fluidly connected to each compartment of the first and second compartments 102 and 104 in a packaged state of dispensing device 100. Indeed, for optimal preservation of the hair treatment formulation during storage, each compartment of the first and second compartments 102 and 104 is sealed off from the other, for example, by way of independent seals or a common seal independently sealing each of the foregoing compartments. That said, the mixing tip 106 can still be coupled to a remainder of the dispensing device 100, thereby demonstrating in the packaged state of the dispensing device 100 a proper assembly of the dispensing device 100 in the ready-to-dispense state thereof.

Continuing with the pigment-precursor formulation as the oil-based dispersion and the oxidant formulation as the aqueous solution, but with the understanding that the pigment-precursor formulation and the oxidant formulation are not limited to such mixtures, as set forth above, the dispensing device 100 can include the pigment-precursor formulation as the oil-based dispersion in the first compartment 102 of the dispensing device 100 and the oxidant formulation as the aqueous solution in the second compartment 104 of the dispensing device 100. Again, each compartment of the first and second compartments 102 and 104 is sealed off from the other for optimal preservation of the hair treatment formulation in the packaged state of the dispensing device 100. However, upon unsealing each of the foregoing compartments and fluidly connecting the mixing tip 106 thereto, thereby instantiating the ready-to-dispense state of the dispensing device 100, the dispensing device 100 is configured for combining the oil-based dispersion and the aqueous solution in at least the mixing tip 106 to form the emulsion including the pigment for hair treatment by way of a dispensing action corresponding to the dispensing device 100.

FIG. 2 illustrates a double-barreled syringe 108 with the mixing tip 106 as the dispensing device 100 in accordance with some embodiments.

When the dispensing device 100 is the double-barreled syringe 108, the first compartment 102 of the dispensing device 100 corresponds to a first barrel chamber defined by a first barrel 110 of the syringe 108, the first barrel chamber including the pigment-precursor formulation as the oil-based dispersion. Likewise, the second compartment 104 of the dispensing device 100 corresponds to a second barrel chamber defined by a second barrel 112 of the syringe 108 conjoined with the first barrel 110, the second barrel chamber including the oxidant formulation as the aqueous solution. Such a double-barreled syringe 108 also includes a plunger 114 having a pair of plunger shafts 116 with a common plunger flange 118 but independently tipped with a pair of pistons (not shown) distributed therebetween. If necessary, the plunger 114 can be clipped or otherwise held in place relative to the first and second barrels 110 and 112 of the syringe 108 in the packaged state thereof. Upon instantiating the ready-to-dispense state of the dispensing device 100, as set forth above, the double-barreled syringe 108 is configured for combining the oil-based dispersion and the aqueous solution in at least the mixing tip 106 to form the emulsion by way of pushing the plunger 114 and, thus, both plunger shafts 116 simultaneously into the first and second barrels 110 and 112 of the double-barreled syringe 108.

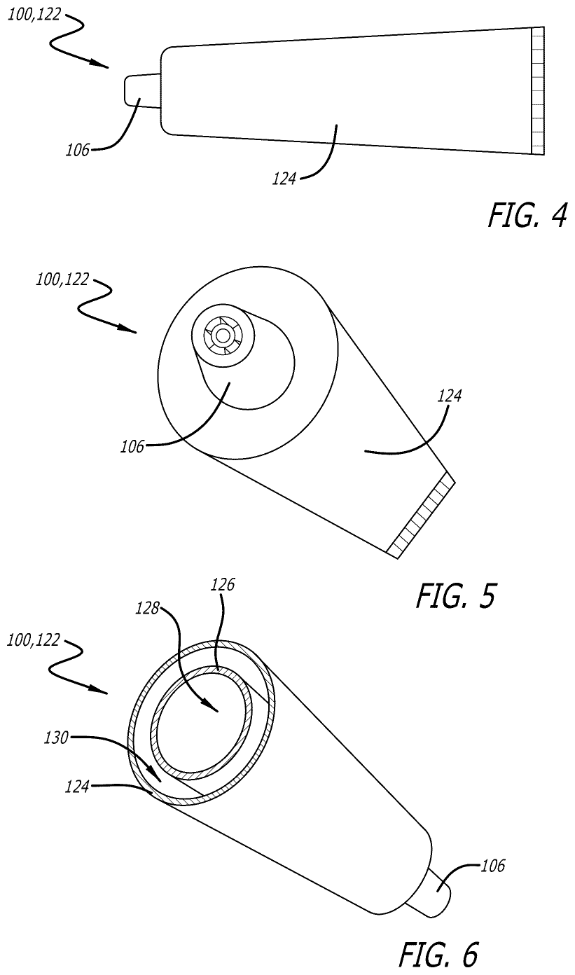

FIG. 3 illustrates a double-compartmented bag 120 with the mixing tip 106 as the dispensing device 100 in accordance with some embodiments. FIGS. 4-6 illustrate a double-compartmented tube 122 with the mixing tip 106 as the dispensing device 100 in accordance with some embodiments.

When the dispensing device 100 is the double-compartmented bag 120 or tube 122, each compartment of the first and second compartments 102 and 104 can be defined by an external piece 124 substantially independent of the other (even if initially formed from the same piece) as well as an internal piece 126 that divides the first and second compartments 102 and 104. Such pieces 124 and 126 can be bonded, crimped, or otherwise held together along a length of the dispensing device 100 as well as along a sealed end of the dispensing device 100 opposite a dispensing end thereof, thereby forming the first and second compartments 102 and 104 shown in FIG. 3. Alternatively, one compartment, namely an inner compartment 128, of the first and second compartments 102 and 104 can be disposed in the other compartment, namely an outer compartment 130, of the first and second compartments 102 and 104 as best shown in FIG. 6, which illustrates the dispensing device 100 prior to sealing the sealed end of the dispensing device 100. The outer compartment 130 of the first and second compartments 102 and 104 is defined by an external piece 124 around both the first and second compartments 102 and 104, and the inner compartment 128 of the first and second compartments 102 and 104 is defined by an internal piece 126 around it only. Whether the double-compartmented bag 120 or tube 122, the dispensing device 100 can include the pigment-precursor formulation as the oil-based dispersion in the first compartment 102 of the dispensing device 100 and the oxidant formulation as the aqueous solution in the second compartment 104 of the dispensing device 100 or vice versa. Upon instantiating the ready-to-dispense state of the dispensing device 100, as set forth above, the double-compartmented bag 120 or tube 122 is configured for combining the oil-based dispersion and the aqueous solution in at least the mixing tip 106 to form the emulsion by way of squeezing the bag 120 or tube 122 from the sealed end toward the dispensing end thereof.

FIGS. 7 and 8 illustrate a double-compartmented pouch 132 with a tear-off opening strip 134 in accordance with some embodiments.

When the dispensing device 100 is the double-compartmented pouch 132, each compartment of the first and second compartments 102 and 104 can be defined by at least a perimeter seal 136 and an internal seal 138 that divides the first and second compartments 102 and 104. Such seals 136 and 138 can be heat sealed, bonded, crimped, or otherwise held together along a length of the double-compartmented pouch 132 as well as along a sealed end 140 of the double-compartmented pouch 132 opposite a dispensing end 142 thereof, thereby forming the first and second compartments 102 and 104 shown in FIGS. 7 and 8. While not shown, the sealed end 140 of the double-compartmented pouch 132 can include a gusset configured to provide additional inner space or volume to hold the pigment-precursor formulation. In addition, the gusset can be configured to provide a base or support for the double-compartmented pouch 132 when the gusset is opened, thereby allowing the double-compartmented pouch 132 to stand upright on a surface. The dispensing end 142 of the double-compartmented pouch 132 includes the tear-off opening strip 134, which is configured to be torn off the double-compartmented pouch 132 for dispensing the pigment-precursor formulation. Notably, the double-compartmented pouch 132 of FIG. 7 and that of FIG. 8 differ with respect to the neck 144 between the tear-off opening strip 134 and the shoulders 146 of the double-compartmented pouch 132 of FIG. 7. Such a neck 144 functions to decrease flow rate and enhance laminar flow, whereas the lack of the neck 144 in the double-compartmented pouch 132 of FIG. 8 functions to increase flow rate and enhance turbulent flow. Whether the double-compartmented pouch 132 of FIG. 7 or that of FIG. 8, the double-compartmented pouch 132 can include the pigment-precursor formulation as the oil-based dispersion in the first compartment 102 and the oxidant formulation as the aqueous solution in the second compartment 104 of the double-compartmented pouch 132 or vice versa. Upon tearing off the tear-off opening strip 134, the double-compartmented pouch 132 is configured for combining the oil-based dispersion and the aqueous solution to form the emulsion by way of squeezing the double-compartmented pouch 132 from the sealed end toward the dispensing end thereof and into a non-reactive receptacle as set forth in the method below.

Notably, the double-compartmented pouch 132 includes a front side and a back side of a same or different flexible construction. For example, each side of the front side and the back side can be a laminate of one or more layers of polyester, nylon, aluminum, polypropylene, or polyethylene. The polyester can provide a glossy, rigid layer that can be printed on, the nylon can provide puncture resistance, the aluminum can provide a thin, effective gas barrier, the polypropylene can be used as a sealing layer, and the polyethylene can be used instead of polypropylene as the sealing layer, as well as a bonding layer.

Notwithstanding the foregoing, it should be understood the hair treatment formulation can be alternatively distributed between any combination of two single-compartmented dispensing devices selected from syringes, bags, tubes, or the like. Indeed, a first syringe, a first bag, or a first tube can include the pigment-precursor formulation as the oil-based dispersion, and a second syringe, a second bag, or a second tube, which need not be respective to the first syringe, the first bag, or the first tube, can include the oxidant formulation as the aqueous solution. Upon unsealing the two single-compartmented dispensing devices, the dispensing devices are configured for dispensing the oil-based dispersion and the aqueous solution in a non-reactive receptacle for mixing to form the emulsion including the pigment for hair treatment.

### Hair Treatment Kits

A hair treatment kit includes at least one dispensing device 100 including the hair treatment formulation disposed in the dispensing device 100. In addition, the hair treatment kit can include one or more additional components selected from one or more packages of chelating shampoo for removing at least transition metals or other residues from hair prior to applying the emulsion, one or more tint brushes for applying the emulsion, gloves for preventing hands from becoming stained by the emulsion, and instructions for using the hair treatment kit.

### Methods

Methods include methods of making and using any one or more components of the hair treatment kit up to an entirety of the hair treatment kit. In an example, the methods can include a method of making the hair treatment formulation. In another example, the methods can include a method of using the hair treatment kit.

Continuing with the pigment-precursor formulation as the oil-based dispersion and the oxidant formulation as the aqueous solution, the method of making the hair treatment formulation can include making the oil-based dispersion and the aqueous solution, as set forth below.

When making the oil-based dispersion, the method can include mixing the one or more stabilizers into the one or more oils, optionally, with heating, as needed. For example, the method can include mixing dextrin palmitate into coconut oil with heating to ensure the coconut oil remains a liquid for the mixing. The method can also include subsequently mixing any single constituent or combination of constituents selected from the pigment precursor, the one or more transition metal catalysts, and the one or more pH-modifying agents into a nascent oil-based dispersion of the one or more stabilizers in the one or more oils, thereby forming the oil-based dispersion of the hair treatment formulation. For example, the method can include subsequently mixing an admixture including levodopa, sodium bicarbonate, and manganese gluconate into the oil including the dextrin palmitate to form the oil-based dispersion of the hair treatment formulation. However, should the oil-based dispersion include the one or more preservatives or the one or more haircare ingredients as set forth above, such constituents can be mixed into the one or more oils, the oil including the one or more stabilizers, or that including the pigment precursor, the one or more transition metal catalysts, or the one or more pH-modifying agents, as the case might be.

When making the aqueous solution, the method can include mixing the oxidant into water. For example, the method can include mixing hydrogen peroxide (e.g., 3% aqueous hydrogen peroxide) into the water. The method can also include subsequently mixing the one or more viscosity modifiers into the aqueous solution including the oxidant, thereby forming the aqueous solution of the hair treatment formulation. For example, the method can include subsequently mixing konjac gum into the aqueous solution including the hydrogen peroxide to form the aqueous solution of the hair treatment formulation. However, should the aqueous solution include the one or more preservatives or the one or more haircare ingredients as set forth above, such constituents can be mixed into the water, the aqueous solution including the oxidant, or that including the one or more viscosity modifiers, as the case might be.

Notably, in a method of preparing the dispensing device 100, each of the oil-based dispersion and the aqueous solution of the hair treatment formulation can be loaded into its corresponding compartment 102 or 104 of the dispensing device 100 and sealed therein.

The method of using the hair treatment kit can include washing hair with a chelating shampoo to remove at least transition metals or other residues from the hair prior to applying any of the emulsion to the hair. When using the double-compartmented dispensing device 100, the method also includes dispensing the emulsion including the pigment for hair treatment from the mixing tip 106 of the dispensing device 100. While the oil-based dispersion and the aqueous solution form the emulsion and at least some of the pigment in the mixing tip 106 during the dispensing from the dispensing device 100, it can be beneficial to dispense the emulsion into a non-reactive receptacle to further mix it with, for example, the tint brush, and allow oxidation of the pigment precursor to the pigment to complete before application. When using two single-compartmented dispensing devices, however, the method includes dispensing the oil-based dispersion and the aqueous solution in such a non-reactive receptacle and mixing them, for example, with the tint brush, to form the emulsion. No matter the dispensing device or devices, the method also includes applying the emulsion to hair or spreading the emulsion in the hair with the tint brush such as by iterative transfer from the foregoing receptacle to effectuate the hair treatment with at least the pigment. Lastly, the method also includes rinsing the emulsion from the hair subsequent to a period of treatment time (e.g., 10 minutes).

### EXAMPLES

### Example 1: Dark Brown Formulation

As the oil-based dispersion of the hair treatment formulation, 1.40 g dextrin palmitate is stirred into 33.52 g of coconut oil while heating between 80 and 130° C. Upon cooling, an admixture of 17.28 g of M. pruriens, 1.8 g of manganese gluconate, and 15.84 g of sodium bicarbonate is stirred into the oil-based dispersion of dextrin palmitate and coconut oil to form the oil-based dispersion of the hair treatment formula.

As to the aqueous solution of the hair treatment formulation, 33.6 g of 3% hydrogen peroxide is added to 80 g of distilled water followed by 1.44 g of konjac gum. An overhead mixer at about 1200 RPM is used to mix the resulting aqueous solution for an hour to form the aqueous solution of the hair treatment formulation.

When the oil-based dispersion and the aqueous solution of the dark brown hair treatment formulation are combined to form an emulsion and applied to hair, as in the method set forth above, the color imparted or restored to the hair is described as Level 3: Dark brown in accordance with the hair color level system used across various professional hairdressing brands.

### Example 2: Light Brown Formulation

As the oil-based dispersion of the hair treatment formulation, 1.40 g dextrin palmitate is stirred into 33.52 g of coconut oil while heating between 80 and 130° C. Upon cooling, an admixture of 18.58 g of M. pruriens, 0.43 g of ferrous gluconate, 3.21 g of sodium bicarbonate, and 12.70 g of sodium carbonate is stirred into the oil-based dispersion of dextrin palmitate and coconut oil to form the oil-based dispersion of the hair treatment formula.

As to the aqueous solution of the hair treatment formulation, 33.6 g of 3% hydrogen peroxide is added to 80 g of distilled water followed by 1.44 g of konjac gum. An overhead mixer at about 1200 RPM is used to mix the resulting aqueous solution for an hour to form the aqueous solution of the hair treatment formulation.

When the oil-based dispersion and the aqueous solution of the light brown hair treatment formulation are combined to form an emulsion and applied to hair, as in the method set forth above, the color imparted or restored to the hair is described as Level 5: Light brown in accordance with the hair color level system used across various professional hairdressing brands.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A hair treatment formulation, comprising:
a dispersion including:
a continuous phase including one or more oils; and
a dispersed phase including:
a pigment precursor; and
one or more transition metal catalysts for increasing an oxidation rate of the pigment precursor; and
an aqueous solution including an oxidant for oxidizing the pigment precursor to a pigment,
wherein the dispersion and the aqueous solution form an oil-in-water emulsion for hair treatment upon combination with each other, the emulsion including the pigment.

2. The hair treatment formulation of claim 1, wherein the one or more oils are selected from coconut oil, fractionated coconut oil, olive oil, argan oil, avocado oil, jojoba oil, sweet almond oil, grapeseed oil, baobab oil, castor oil, macadamia nut oil, marula oil, and sea buckthorn oil.

3. The hair treatment formulation of claim 1, wherein the pigment precursor is present in the dispersed phase as a constituent of Mucuna pruriens seed powder or an extract thereof.

4. The hair treatment formulation of claim 1, wherein the one or more transition metals are selected from manganese and iron.

5. The hair treatment formulation of claim 1, the dispersed phase of the dispersion further including one or more stabilizers for keeping the dispersed phase dispersed in the continuous phase, wherein the one or more stabilizers are selected from dextrin palmitate, sucrose laurate, and dibutyl lauroyl glutamide.

6. The hair treatment formulation of claim 1, the dispersed phase of the dispersion further including one or more pH-modifying agents in a concentration suitable for modifying the pH of an aqueous phase of the emulsion to be greater than pH 7 upon combination of the dispersion and the aqueous solution.

7. The hair treatment formulation of claim 1, wherein the oxidant is hydrogen peroxide.

8. The hair treatment formulation of claim 1, wherein the dispersion, the aqueous solution, or both the dispersion and the aqueous solution independently include one or more preservatives selected from phenethyl alcohol, sodium benzoate, gluconolactone, potassium sorbate, sodium anisate, sodium phytate, aspen bark extract, willow bark extract, leucidal liquid, propanediol, pentylene glycol, glycerin, ethylhexyl glycerin, caprylyl glycol, and glyceryl caprylate-caprate.

9. A hair treatment kit, comprising:
a dispensing device including a first compartment and a second compartment;
a dispersion in the first compartment, the dispersion including:
a continuous phase including one or more oils; and
a dispersed phase including:
a pigment precursor; and
one or more transition metal catalysts for increasing an oxidation rate of the pigment precursor; and
an aqueous solution in the second compartment, the aqueous solution including an oxidant for oxidizing the pigment precursor to a pigment,
wherein the dispersion and the aqueous solution form an oil-in-water emulsion for hair treatment upon dispensation from the dispensing device and combination with each other, the emulsion including the pigment.

10. The hair treatment kit of claim 9, wherein the one or more oils are selected from coconut oil, fractionated coconut oil, olive oil, argan oil, avocado oil, jojoba oil, sweet almond oil, grapeseed oil, baobab oil, castor oil, macadamia nut oil, marula oil, and sea buckthorn oil.

11. The hair treatment kit of claim 9, wherein the pigment precursor is present in the dispersed phase as a constituent of Mucuna pruriens seed powder or an extract thereof.

12. The hair treatment kit of claim 9, wherein the one or more transition metal catalysts respectively include one or more transition metals selected from those in a range of the Periodic Table of the Elements defined by Groups 7-12 and Periods 4 and 5.

13. The hair treatment kit of claim 9, the dispersed phase of the dispersion further including one or more stabilizers for keeping the dispersed phase dispersed in the continuous phase, the one or more stabilizers selected from dextrin palmitate, sucrose laurate, and dibutyl lauroyl glutamide.

14. The hair treatment kit of claim 9, the dispersed phase of the dispersion further including one or more pH-modifying agents in a concentration suitable for modifying the pH of an aqueous phase of the emulsion to be greater than pH 7 upon combination of the dispersion and the aqueous solution.

15. The hair treatment kit of claim 9, wherein the oxidant is hydrogen peroxide.

16. The hair treatment kit of claim 9, the dispensing device further including a mixing tip to which each compartment of the first and second compartments is fluidly connected in a ready-to-dispense state of the dispensing device such that dispensation from the dispensing device through the mixing tip combines the dispersion and the aqueous solution to form the emulsion.

17. The hair treatment kit of claim 16, wherein the dispensing device is a double-barreled syringe, the first compartment of the dispensing device corresponding to a first barrel chamber defined by a first barrel of the syringe, and the second compartment of the dispensing device corresponding to a second barrel chamber defined by a second barrel chamber of the syringe.

18. The hair treatment kit of claim 16, wherein the dispensing device is a double-compartmented tube or bag in which each compartment of the first and second compartments is defined by an external piece substantially independent of the other and an internal piece dividing the first and second compartments.

19. The hair treatment kit of claim 16, wherein the dispensing device is a double-compartmented tube or bag in which one compartment of the first and second compartments is disposed in the other compartment of the first and second compartments.

20. The hair treatment kit of claim 9, further comprising one or more additional components selected from a package of chelating shampoo for removing at least transition metals or other residues from hair prior to applying the emulsion, a tint brush for applying the emulsion, and instructions for using the hair treatment kit.
